(19) United States
(12) Plant Patent Application Publication
(10) Pub. No.: US 2013/0318666 P1
(43) Pub. Date: Nov. 28, 2013

(54) Variety of hydrangea plant named 'Stramini'

(50) ;

(71) Applicants: Jan van Straaten, Harderwijk (NL); Johan van Straaten, Harderwijk (NL)

(72) Inventors: Jan van Straaten, Harderwijk (NL); Johan van Straaten, Harderwijk (NL)

(21) Appl. No.: 13/506,883

(22) Filed: May 22, 2012

(51) Int. Cl. A01H 5/00

(52) U.S. Cl. PLT/250

(57) ABSTRACT

A new and distinct Hydrangea plant having multicolored flowers and strong stems.

### BOTANICAL CLASSIFICATION

Hydrangea macrophylla

### VARIETAL DENOMINATION

‘STRAMINI’

### BACKGROUND OF THE INVENTION

The present invention comprises a new and distinct cultivar of Hydrangea plant known by the varietal name ‘Stramini’. The new variety was discovered in 2006 in a planned breeding program in The Netherlands. The purpose of the breeding program was to develop Hydrangeas with improved green-like flower colors. The new variety is the result of a cross between breeding selection Seedling 1012 (female parent, unpatented) and Seedling HY 2586 (male parent, unpatented). The compact nature of ‘Stramini’ is similar to its female parent, but it differs from Seedling 1012 in flower color. The flower color of ‘Stramini’ is similar to its male parent, but it differs from Seedling HY 2586 in leaf shape. The new variety was first asexually reproduced in 2007 via propagation by cuttings in The Netherlands. The new variety has been trial and field tested in The Netherlands and has been found to retain its distinctive characteristics and remain true to type through successive propagations.

The following traits distinguish ‘Stramini’ as a new and distinct cultivar from other Hydrangea varieties known to the breeder:

1. Green-like flowers; and

2. Strong stems.

### DESCRIPTION OF THE DRAWINGS

The accompanying photographic drawings illustrate the new variety, with the color being as nearly true as is possible with color illustrations of this type:

FIG. 1 is photograph of an entire plant of the new variety; and

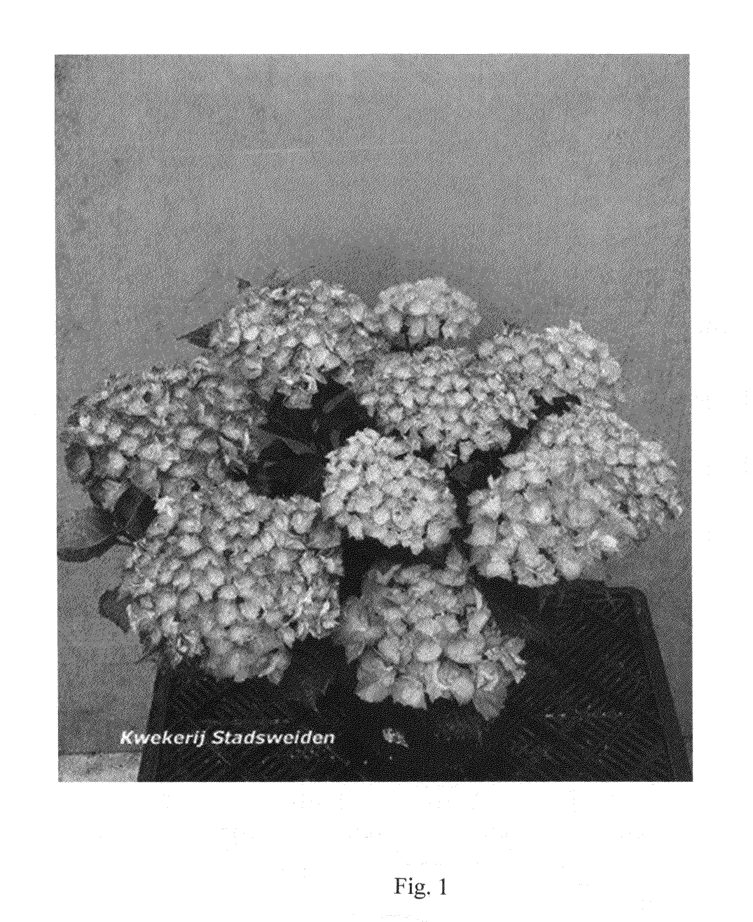

FIG. 2 is another photograph of an entire plant of the new variety.

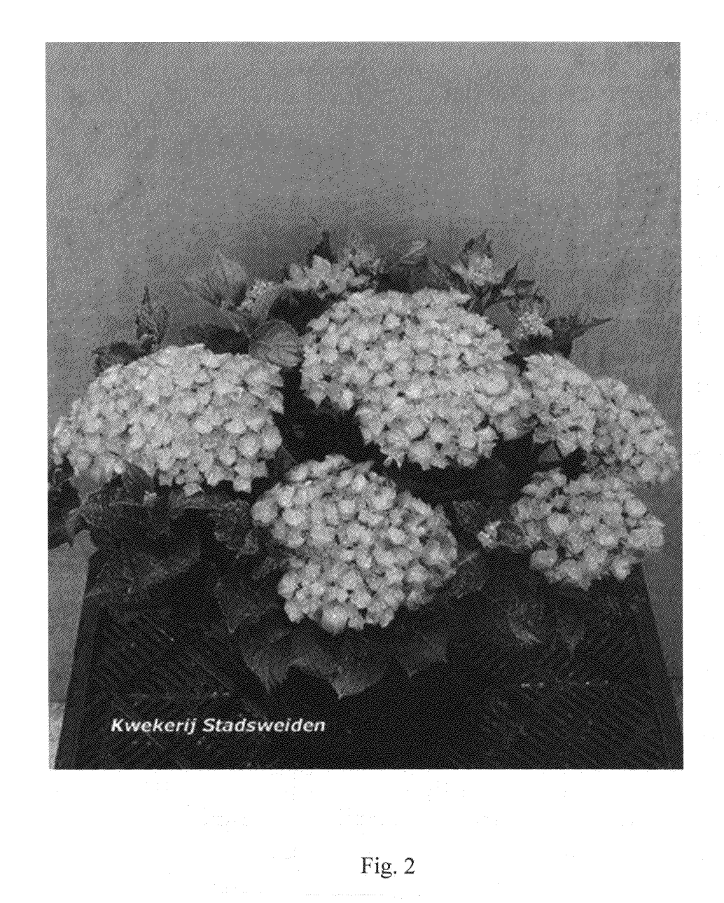

### DESCRIPTION OF THE PLANT

The following detailed description sets forth characteristics of the new cultivar. The plants were grown in 15 cm. pots, 8 per m², in ordinary potting soil with granulated fertilizers at a pH of 6.0. The plants were two years old when described. Measurements and color readings of the plants were taken in The Netherlands under natural light. Color references are primarily to the HTML Color Chart.
Plant:
   Time to initiate roots.—About 14 days at 22° C.
   Time to develop roots.—About 18 days at 18-20° C.
   Rooting habit.—Moderate.
   Time to produce a finished flowering plant from a rooted cutting.—About 48 weeks in a 15 cm. container.
   Form.—Mounded.
   Height (from media surface to top of foliage).—35-40 cm.
   Height (from media surface to top of flowers).—40-45 cm.
   Plant diameter.—35-40 cm.
   Vigor.—Good.
   Strength.—Good.
   Branching habit.—Moderately-branched.
   Trunk.—Main trunk length: 8 cm. Diameter: 1 cm. Color: Green. Texture: Shiny. Pubescence: Absent. Internode length: 1.5-2 cm.
   Lateral branches.—Length: Young: 5-10 cm. Mature: 25-30 cm. Diameter: Young: 0.5 cm. Mature: 0.5 cm. Internode length: 6 cm. Texture: Shiny. Pubescence: Absent. Color: Young: Chartreuse 7F FF 00. Mature: Dark Green 00 64 00.
   Foliage.—Arrangement: Single. Stipules: None present. Size of leaf: Length: 12 cm. Width: 7-8 cm. Shape of leaf (generally): Young: Elliptic/obtuse. Mature: Elliptic/obtuse. Shape of apex: Lanceolate. Shape of base: Round. Texture: Shiny. Aspect: V-formed. Margin type: Serrate. Pubescence: None present. Color: Young leaves: Upper surface: Forest Green 22 8B 22. Lower surface: Forest Green 22 8B 22. Mature leaves: Upper surface: Dark Green 00 64 00. Lower surface: Dark Green 00 64 00. Petiole: Shape: Square. Length: 2 cm. Diameter: 0.5 cm. Color: Green. Veins: Type: Cross-venulate. Color: Upper surface: Olive 80 80 00. Lower surface: Olive 80 80 00.
Flower:
   Natural flowering season.—May until October in Harderwijk, The Netherlands.
   Blooming habit.—Continuous from late spring through summer.
   Bud (measurements made when cluster size was 3 cm.).—Shape of cluster: Round. Diameter of cluster: 3 cm. Number of buds per cluster: 40-50. Shape: Ball. Length: 0.3 cm. Width: 0.3 cm.
   Inflorescence.—Shape: Round. Length: 16-20 cm. Diameter: 16-20 cm. Average number of flowers per inflorescence: 40. Typical number of inflorescences per plant: 6. Lastingness of an individual flower: 8 weeks. Lastingness of an inflorescence: 8 weeks.
   Petals.—Number per flower: 5. Color: Upper surface: Pink FF CO CB. Lower surface: Pink FF CO CB. Shape: Lanceolate and attenuate. Length: 0.3 cm. Width: 0.3 cm. Apex: Triangular. Base: Flat. Margin: Erect. Texture: Shiny. Tonality from a distance: Full.
   Petaloids.—Quantity: 5. Shape: Pointed. Length: 2 cm. Width: 3 cm. Color: Upper surface: Multicolored; Hot Pink FF 69 B4, Pale Violet Red DB 70 93, and Olive 80 80 00. Lower surface: Multicolored; Hot Pink FF 69 B4 and Olive 80 80 00.
   Pedicels.—Length: 2 cm. Diameter: 0.2 cm. Color: Multicolored; Hot Pink FF 69 B4 and Olive 80 80 00.
   Peduncles.—Length: 3-4 cm. Diameter: 0.3-0.4 cm. Texture: Smooth. Color: Olive 80 80 00.
   Stamens.—Number per flower: 10. Filament: Length: 0.3 cm. Color: White FF FA FO. Anthers: Shape: Elongated oval. Length: 0.2 cm. Color: Medium Purple 93 70 DB. Pollen: Color: Beige F5 F5 DC. Amount: Abundant.
   Pistils.—Number: 3. Length: 0.3 cm. Style: Length: 0.2 cm. Color: Light Pink FF B6 C1. Stigma: Shape: Long and oval. Color: White. Size: 0.15 cm. Ovaries (superior): Length: 0.2 cm. Width: 0.2 cm. Color: Beige F5 F5 DC.
   Fragrance.—None observed.
   Disease resistance.—Tolerant to Botrytis.
   Temperature tolerance.—Very good as long as direct sunlight is limited.
   Drought tolerance.—Medium.

I claim:

1. A new and distinct variety of Hydrangea plant substantially as shown and described herein.
